(19) United States
(12) Patent Application Publication
(10) Pub. No.: US 2006/0231106 A1
(43) Pub. Date: Oct. 19, 2006

(54) Medical backboard apparatus

(76) Inventor: Steven Barron Ostrowski, Hamilton (CA)

Correspondence Address: BEUSSE WOLTER SANKS MORA & MAIRE, P. A., 390 NORTH ORANGE AVENUE, SUITE 2500, ORLANDO, FL, 32801, US

(21) Appl. No.: 11/335,864

(22) Filed: Jan. 19, 2006

Related U.S. Application Data
(60) Provisional application No. 60/645,585, filed on Jan. 20, 2005.

(51) Int. Cl. A61B 19/00

(52) U.S. Cl. 128/869

(57) ABSTRACT

A backboard for medical use for supporting and transporting a patient comprises a flat board formed of a fluted plastic sheet material and having a plurality of handholds distributed along opposite longitudinal edges. A pair of strengthening members formed of fluted plastic sheet material and having a plurality of lengthwise extending fold lines enabling folding of the members into triangular shapes with laterally extending flaps are bonded to an underside of the flat board via the flaps. A plurality of slots are formed through the strengthening members and aligned for passing straps for holding a patient on the backboard. In another form, the backboard comprises a flat board having a pair of lengthwise extending side members defined by grooves cut partially through the board and extending the length of the board. At least one slot passes through each of the side members at a common distance from one end of the board and a strap extends through the slot in each side member and is adapted for pulling the side members toward each other along an underside of the board. The grooves have a width to prevent the side members from being pulled more than ninety degrees from a plane of the board.

This application claims the benefit of U.S. provisional application No. 60/645,585, filed Jan. 20, 2005.

The present invention relates to a medical backboard apparatus and more particularly to a backboard apparatus which can be initially stored in a substantially flat configuration and is formed from lightweight plastic sheeting.

### BACKGROUND OF THE INVENTION

U.S. Pat. No. 6,094,761 discloses a disposable backboard for carrying a patient from a folded blank. The blank is stored flat and is folded at the time that it is needed to form the backboard. The described backboard is intended to be disposable and is made from a coated paper or cardboard material. The finished product includes a plurality of hand grips that are located on one of the triangular shaped supports under the backboard.

The backboard of the '761 patent is made from a single sheet of cardboard and requires a relatively wide area for storage. In addition, the handles for the backboard are under the top surface of the backboard in one of the lengthwise extending triangular supports. In addition, the backboard is intended for a one-time use.

### BRIEF DESCRIPTION OF THE DRAWINGS

FIG. 1 is a bottom perspective view of the medical backboard in a partially configured arrangement;

FIG. 2 is a bottom perspective view of the medical backboard of FIG. 1 in an assembled configuration;

FIG. 3 is a bottom perspective view of the medical backboard of FIG. 1 showing one method of final assembly of the backboard;

FIG. 4 is a top perspective view of the backboard of FIG. 1 showing positioning of the patient straps and a head clamp;

FIG. 5 is a perspective view of the head clamps in the backboard of FIG. 4 in an unassembled configuration;

FIG. 6 illustrates the assembled head clamps positioned with respect to a patient on the backboard of FIG. 1;

FIG. 7 is another view of the head clamps arrangement showing both head clamps positioned for supporting a patient's head;

FIGS. 8A and 8B are plan views and end views of the backboard of FIG. 1 prior to folding of the side support members;

FIGS. 9A and 9B are bottom plan and end views of the backboard of FIG. 1 showing the side members folded for storage;

FIGS. 10A and 10B are bottom plan and end view of the backboard of FIG. 1 in a final assembled configuration;

FIG. 11 is a perspective view of an alternate embodiment of a medical backboard;

FIG. 12 is a perspective view of the backboard of FIG. 11 in a final assembled configuration; and

FIG. 13 is an end view of the backboard of FIG. 12.

### DETAILED DESCRIPTION OF THE INVENTION

The present invention is produced using a plastic fluted board such as the type available from Coroplast, Inc. under their trademark Corplast® or from Matra Plast Industries, Inc. under their trademark Hi-core®. The material has a finished upper and lower surface which can be cleaned but at the same time the fluted intermediate structure between the upper and lower surface has a plurality of open cells that make the finished product relatively lightweight. In a preferred embodiment, the backboard of the present invention is manufactured from such plastic fluted board having a ten millimeter thickness. This thickness is chosen because such a board is a commercially available board that can be obtained without special orders or special manufacturing processes. In addition, applicant's improved backboard has substantial lifting strength and is capable of holding a five hundred pound load while at the same time being easy to handle and inexpensive. Further, use of the fluted plastic makes the backboard lightweight and waterproof so that it can be cleaned and re-used. The use of the fluted plastic also provides sufficient strength so that the handles can be formed in the top surface of the backboard to simplify and handling of the assembled backboard and avoid the awkward lifting positions in the prior art.

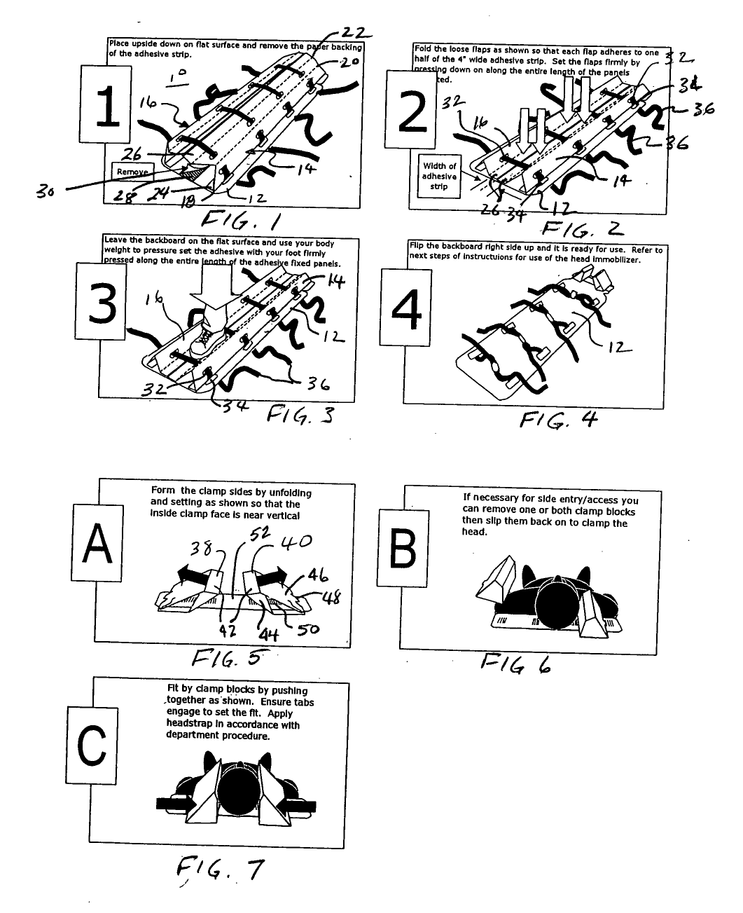

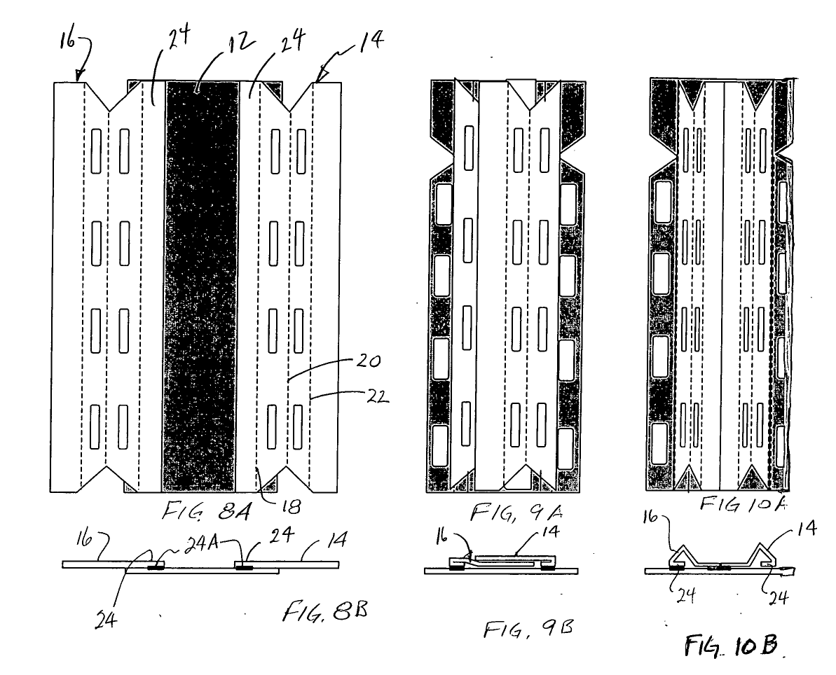

Referring to FIGS. 1-3 and 4 together, the backboard apparatus of the present invention is shown in FIG. 1 as being initially formed from three separate pieces of thin wall fluted plastic sheeting or other similar type sheet material that is lightweight and has strengths and water or rot resistance. The backboard 10 is shown in an inverted position in FIG. 1 and comprises a top sheet 12 and a pair of side sheets 14 and 16. Each of the side sheets 14, 16 are pre-cut to form lengthwise slots to enable the sheets to be folded at precise locations. Typically, the sheets are cut with a small router bit so as to form a slot in one wall of the twin wall plastic sheeting so as to allow the sheeting to be bent or folded along that slot. As shown in FIG. 1, each of the side pieces are cut along three separate lengthwise locations indicated at 18, 20, and 22. The slot along the location 18 defines a first flap 24 that can be adhesively bonded to the top sheet 12. The flap 24 is preferably at least about two inches in width and extends the length of the board. Flap 24 may be heat welded or adhesively bonded to a bottom surface of sheet 12 to create double thickness and give the backboard the required strength. The remainder of the side member 14 is foldable about the fold line 18 to an extent greater than 90 degrees so that when placed in a final desired position, the side member 14 forms a triangular configuration as shown in FIG. 3. The peak of the triangle configuration is defined by the fold line 20. The fold line 22 defines another flap portion 26 which can be adhesively bonded to the bottom panel 12 to complete the triangle configuration of the side member as is illustrated in FIGS. 2, 3. The initial assembly of the board at the time of use is achieved by folding the side member 14 along the different fold lines 18, 20, 22 and then adhesively bonding the remaining flap 26 to the bottom of the top member 12. Preferably, the backboard 10 or the flaps 26 are provided with a strip of adhesive material 28 that is normally protected by a releasable cover 30 that can be peeled up at the time it is desired to attach the flap 26 to the bottom surface of the upper member 12. The side member 16 is folded and attached to the member 12 in the same manner as the side member 14.

As shown in FIG. 2, once the side members 14, 16 have been folded along the fold lines 18, 20, 22, the flaps 26 are then adhesively bonded in place so that each of the side members 14, 16 form the pair of triangle shaped support members for the top member 12. FIG. 3 illustrates a final step in bonding the flaps 26 to the top member 12 by simply stepping or walking along the abutting flaps 26 from each of the side members to firmly bond the flaps to the member 12. The board 10 is completed by a plurality of straps 32 which extend through slots 34 spaced along the length of the two triangle shaped supports 14, 16 and pass through slots 36 in the top member 12. As shown in FIG. 4, the straps meet across the top surface of the member 12 and can be used to hold a patient positioned on the board in a fixed position.

The board 10 may also be provided with head immobilizers as shown in FIGS. 5, 6, and 7. As illustrated in FIG. 6, the head immobilizers are also formed of thin wall fluted plastic sheet or other sheet material that can be folded flat when not in use. When needed, the head immobilizers indicated at 38, 40 are raised from the flat storage position to form the shape as shown in FIG. 7. Each of the clamps 38 and 40 are made out of folded sections of the twin wall fluted plastic sheeting or other similar structural sheet material and can be characterized as four-sided, open end boxes. Each clamp has a relatively narrow upper segment 42 and a pair of depending segments 44, 46 with the segment 46 being slightly longer than the segment 44. This difference in length creates an angular support out of the segment 46. At the bottom of each segment 46 there is a tab 48 that is configured to fit into one of a plurality of slots 50 formed in a head support 52. The head support 52 may be formed as part of the board member 12 or may be a separate support that is adhesively bonded to the lower support members 14, 16. As shown in FIG. 6, each of the clamps 38, 40 can be positioned on the support 52 after the person's head is in position on the support. The clamp sides 38, 40 are then moved towards the person's head indicated at 54 with the tabs 48 locking into one of the slots 50 to hold the head immobilizer clamps in position as shown in FIG. 7. While the head clamps 38, 40 may be made from the same material as the backboard 12, i.e., thin wall fluted plastic sheet, other plastic sheets could also be used so long as the sheets are capable of bending along predefined fold lines so that the sheets can be folded flat and then raised to create the form shown in FIG. 6 and FIG. 7. It should be noted that the twin wall fluted plastic sheet is well known and is similar to conventional cardboard that is used to form cardboard boxes with the exception of being made from plastic material. It is possible that heavier type cardboard could also be used for this application although such cardboard would not likely be impervious to water damage in the same manner as plastic.

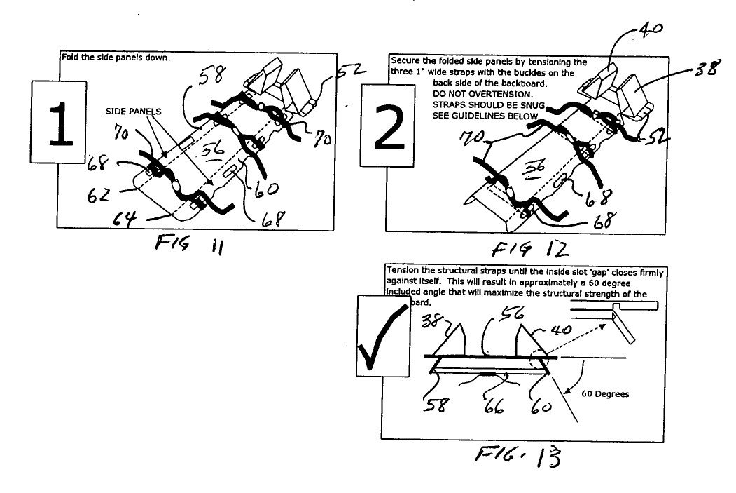

Turning now to FIGS. 8A-10B, there is shown a sequence of different positions of the elements of the inventive backboard during initial manufacture and storage and up to final assembly. In FIG. 8A, there is shown a top plan view of the backboard 12 with each of the side members 14 and 16 laid out in a flat configuration. FIG. 8B is an end view of the assembly of FIG. 8A illustrating the weld portions at 24A which attach the flaps 24 to the board 12. In FIG. 9A, there is shown a top plan view of the side members 14 and 16 being folded over into the storage format to form the compact flat storage as shown in the end view of FIG. 9B. Note that each of the sections of the side members 14 and 16 have the pre-cut fold lines at 18, 20 and 22 which allow the members to be easily formed into the triangular configuration that provides the final strength to the assembled backboard. The advantage of folding as shown in FIGS. 9A and 9B is to achieve a flat storage capacity for a plurality of the backboards. In FIG. 10A, there is shown a bottom plan view of the final configuration of the backboard with the triangular runners formed by the side members 14 and 16. FIG. 10B illustrates an end view of the backboard of FIG. 10A.

Another form of backboard is shown at 56 in FIGS. 11, 12 and 13.

The board 56 is preferentially designed for pediatric use. The board 56 comprises a single unit having a pair of lengthwise slots that are routed in the lower section of the board to enable a pair of side flaps 58 and 60 to be folded downward at an angle. As shown in FIG. 13, it is preferable that the side flaps be folded approximately 60 degrees from the horizontal plane defined by the top of the board 56. As previously described, the slots that are cut along the lines indicated at 62, 64 are preferably formed with a small router bit and cut to a width that allows the flaps 58, 60 to be bent downward 60 degrees before the routed edges of the slot reach contact. For a 10 millimeter thick board, a preferred slot width is about 9 mm. The board shown at 56 includes an additional set of straps indicated at 66 which are provided solely for the purpose of pulling the two side flaps 58, 60 towards each other as shown in FIG. 13. The straps 66 can be drawn just tight enough to hold the side flaps 58, 60 in the desired angular orientation. Each of the flaps 58, 60 are also provided with hand holds indicated at 68 to enable persons to pick up and carry the medical backboard. A separate set of straps indicated at 70 in FIGS. 11 and 12 are used to hold a patient in position on the backboard 56. The backboard 56 may also be provided with the same type of head immobilizer clamps as shown in FIGS. 5, 6, and 7. The clamps are illustrated as being attached to one end of the backboard 56 using the same type of support member 52 either bonded to the backboard 56 or formed as a part of the board 56.

1. A backboard for medical use for supporting and transporting a patient comprising:
a flat board formed of a fluted plastic sheet material and having a plurality of handholds distributed along opposite longitudinal edges thereof;
a pair of strengthening members formed of fluted plastic sheet material and having a plurality of lengthwise extending fold lines enabling folding of the members into triangular shapes with laterally extending flaps, the flaps being bonded to an underside of the flat board to attach the formed triangles thereto.

2. The backboard of claim 1 and including a plurality of slots formed through the strengthening members and aligned for passing straps therethrough for holding a patient on the backboard.

3. A backboard for medical use for supporting a patient thereon comprising:
a flat board having a pair of lengthwise extending side members defined by grooves cut partially through the board and extending the length of the board;
at least one slot passing through each of the side members at a common distance from one end of the board; and
a strap sized and configured for passing through the slot in each side member and adapted for pulling the side members toward each other along an underside of the board, the grooves having a width to prevent the side members from being pulled more than ninety degrees from a plane of the board.
